(19) United States
(12) Patent Application Publication
(10) Pub. No.: US 2007/0276456 A1
(43) Pub. Date: Nov. 29, 2007

(54) Band for Curing Human Body

(76) Inventor: Hwa-Soo Hwang, Suwon-si (KR)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 10/564,906

(22) PCT Filed: Jul. 16, 2004
(86) PCT No.: PCT/KR04/01768

§ 371 Date: Mar. 8, 2007

(30) Foreign Application Priority Data
Jul. 16, 2003 (KR) 10-2003-0048792

(51) Int. Cl. A61N 5/06

(52) U.S. Cl. 607/93; 607/152

(57) ABSTRACT

The present invention is directed to a band for curing a human body, which comprises a flexible sheet member coated with an adhesive on a surface thereof, silver attached to the surface of the sheet member coated with the adhesive, and at least one optical fiber. According to the present invention, the pain, wound or the like can be spontaneously remedied using micro current and light waves such as far infrared rays produced from acupuncture spots, trigger points and around affected parts of the human body. Further, one can easily cure themselves using the band because the band has no side effects and can be simply operated.

### TECHNICAL FIELD

The present invention relates to a band for curing a human body, and more particularly, to a band for curing a human body, which comprises a flexible sheet member with adhesives applied onto a surface thereof, silver attached to the surface of the sheet member with the adhesives applied thereon, and one or more optical fibers.

### BACKGROUND ART

In general, a body region called an acupuncture spot or trigger point is a region where its electrical resistance is relatively low and electrical conductivity is relatively high as compared with the neighboring regions. Thus, curing using a needle for acupuncture has been heretofore made on the region. Recently, medical treatment and equipment using an electrical or electronic needle (e.g., Silver Spike Point), TENS (Transcutaneous Electrical Nerve Stimulation) or EST (Electrical Stimulation for Tissue) have been developed and used in a clinical field. Japanese Patent Laid-open Publication No. (Hei) 7-59867, published on Mar. 7, 1995, has disclosed a patch sheet comprising an adhesive layer applied on a sheet base material and two kinds of powder or granular materials of which electrochemical potentials are different from each other.

However, the curative effect of the conventional medical treatment or therapy using strong current in a mA unit is not excellent because it cannot adjust waveforms of voltages and currents most suitable for disease cure. Worldwide advanced study results show that strong current higher than several mA may rather deteriorate than enhance a physiological activity of an organism and that micro current in a μA unit corresponding to a level produced in an organism is preferred in a physiological activity such as cell regeneration[Refer to “The Body Electric: Electromagnetism and the Foundation of Life” by R. O. Becker and G Seldon, translated by Mindvision Inc., 1995; “Technical paper “Electro-Acuscope/Myopulse Microcurrent” Jin Yang Medical Trading, 1995]. Further, current flowing through a human body often varies according to external factors such as temperature and humidity and internal factors such as mental and physical state, and its waveform also varies on respective regions of the human body. To cure the diseases using electricity, therefore, it is preferable to apply the current having intensity and waveform similar to those of a current produced in an affected part of the human body.

From the foregoing, the inventor of the present invention has filed an application and obtained a patent in Korea(KR) regarding a magnetic patch for remedy wherein a curative effect is enhanced using a micro current produced from the human body through analysis of clinical examples (See Korean Patent No. 221640).

Furthermore, besides the electrical medical treatment, other medical treatment using light waves is also developed. The optical medical treatment includes far-infrared therapy, ultraviolet therapy, photochemical therapy, laser therapy and the like. In particular, it has been verified from a number of documents that the far infrared rays are helpful to the human body. Research and development for the medical instruments or apparatuses using the far infrared rays have been actively performed to a certain extent that the medical instruments or apparatuses can be easily available from the market. Korean Utility Model Registration No. 0213939 discloses a plaster wherein a ball-shaped material comprising, by weight, 80-90% of Tourmaline powder and 10-20% of Chitosan is attached to ventilative nonwoven fabric which is made of cotton and coated with an adhesive. In addition, it has also verified that a small amount of light waves including far infrared rays is emitted from the human body. A device for measuring intensity and distribution of far infrared rays emitted from the human body in order to determine whether the condition of a user is good or bad has been also recently developed.

Nevertheless, integrated and systematic studies on the electrical and optical medical treatment have not yet been made, and thus, the inventor of the present invention has studied a method of efficiently integrating electrical and optical therapy into one and completed the present invention based on clinical experiences made for several years.

### DISCLOSURE OF INVENTION

Accordingly, an object of the present invention is to provide a band for curing a human body wherein diseases such as pain or wound can be spontaneously remedied using micro current and light waves produced from the human body, it has no side effects, and its operation can be simply performed, whereby the general public can easily cure themselves using the band.

The above object of the invention can be accomplished by providing a band for curing a human body which comprises a flexible sheet member coated with an adhesive on a surface thereof, silver attached to the surface of the sheet member coated with the adhesive, and at least one optical fiber.

The band for curing a human body in accordance with the present invention is characterized in that the silver may be a silver foil, silver wire, silver fabric or the combination thereof.

The band for curing a human body in accordance with the present invention is characterized in that the optical fiber is positioned at regular intervals between the sheet member and the silver foil when the silver is made in the form of the silver foil.

The band for curing a human body in accordance with the present invention is characterized in that the optical fiber is alternately arranged on the sheet member at regular intervals with the silver wires when the silver is made in the form of the silver wires.

The band for curing a human body in accordance with the present invention is characterized in that a length of the optical fiber is within a range of 0.1 cm to 2.0 cm.

The band for curing a human body in accordance with the present invention may further comprise a black line disposed on the surface of the sheet member coated with the adhesive.

According to the band of the present invention, the pain, wound or the like can be spontaneously remedied using the micro current and light waves such as far infrared rays produced from acupuncture spots, trigger points and affected parts of the human body. Further, the general public can easily cure themselves using the band because the band can be simply operated.

### BRIEF DESCRIPTION OF DRAWINGS

FIG. 1 is a perspective view of a band for curing the human body wherein a sliver foil and optical fibers are attached to a sheet member according to an embodiment of the present invention.

FIG. 2 is a perspective view of a band for curing the human body wherein a silver foil, optical fibers and black lines are attached to a sheet member and a protective film is further attached to the sheet member with the silver foil, optical fibers and black lines attached thereto according to another embodiment of the present invention.

FIG. 3 is a partially enlarged perspective view illustrating a structure of the band for curing the human body.

### BEST MODE FOR CARRYING OUT THE INVENTION

Hereinafter, preferred embodiments of the present invention will be described in detail with reference to the accompanying drawings.

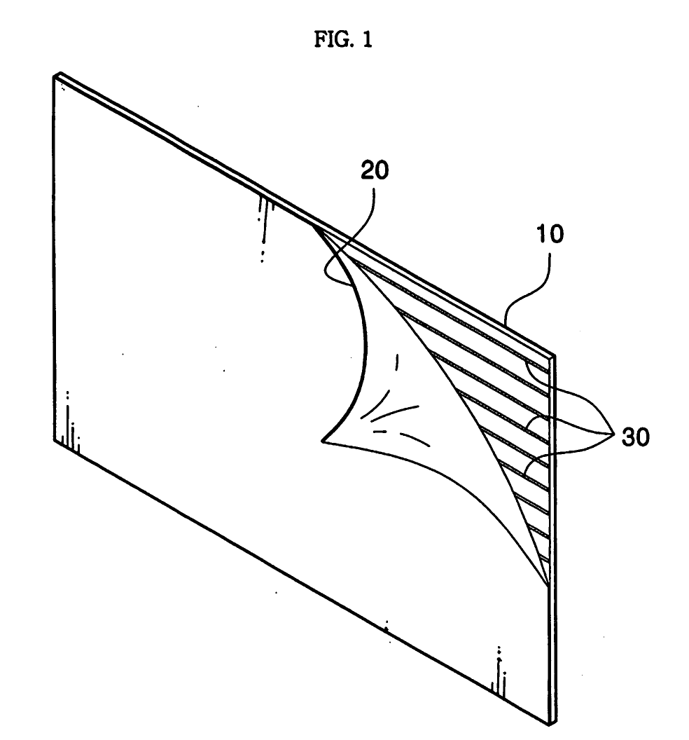

FIG. 1 is a perspective view of an embodiment of a band in accordance with the present invention. As shown in FIG. 1, the embodiment of the present invention is a band for curing the human body which is made by attaching a silver foil 20 and optical fibers 30 onto a flexible sheet member 10 with an adhesive coated onto a surface thereof.

The sheet member 10 serves to attach and secure optical fibers 30 and the silver foil 20 onto a surface thereof. Further, it is preferred that the sheet member 10 be flexible such that the band in accordance with the present invention can be brought into close contact with curved regions of the human body in use. Therefore, it is preferred that the sheet member be made of metal, paper, synthetic resin, fabric, or a material including the aforementioned materials. Particularly, in a case where the material of the sheet member comprises metal, it is preferred that a metal component be gold, silver, platinum, aluminum or alloy thereof. As an adhesive coated on the flexible sheet member, all kinds of adhesives available from the commercial market may be employed but should be made of components harmless to the human body.

The band for curing the human body in accordance with the present invention comprises silver that is attached onto a surface of the sheet member 10 coated with the adhesive. The silver serves to transfer a human current, which is produced around an affected part of the human body, to the affected part of the human body. The silver may be silver powder, silver foil, silver wire, silver fabric or the combination thereof.

In addition, the band for curing the human body in accordance with the present invention comprises one or more optical fibers 30 that are attached to a surface of the sheet member 10 coated with the adhesive. The optical fibers 30 are generally used to transmit light waves to the human body, and particularly, serve to emit the light waves such as far infrared rays produced from the neighboring tissues of affected parts of the patients themselves to their own affected parts. The inventor of the present invention has known from clinical experiences obtained during the repeated traditional Korean medical treatment and studies that the curative effect is much better to use light waves such as far infrared rays emitted from their own bodies of the patients rather than to cure the patients using conventional far infrared therapy equipment. In particular, the curative effect can be maximized by using the light waves such as far infrared rays produced around the affected parts of the patients themselves. Therefore, it is preferred that a length of the optical fiber 30 be within a range of 0.1 cm to 2 cm. It is difficult to manufacture/handle the band and expect a good curative effect when the length of the optical fiber is shorter than 0.1 cm, whereas the curative effect is greatly reduced due to a long distance between the normal tissues emitting the light waves and the affected part absorbing the light waves when the length of the optical fiber is greater than 2.0 cm. More preferably, the length of the optical fiber is shorter than 1.5 cm. Most preferably, the length of the optical fiber is within a range of 0.3 cm to 1.2 cm.

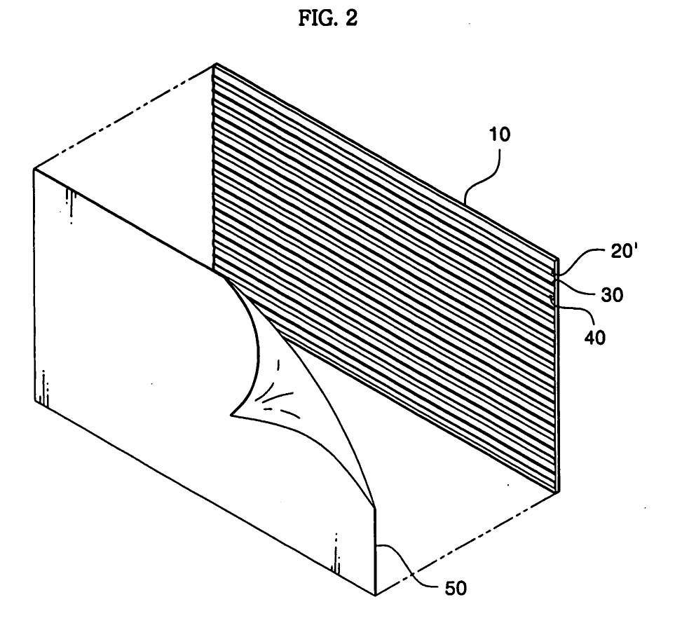

As shown in FIG. 1, in a case where the silver is made in the form of the silver foil 20, the optical fibers 30 are repeatedly arranged at predetermined intervals between the sheet member 10 and the silver foil 20. The resultant band with the optical fibers 30 arranged thereon at the regular intervals may be perforated or divided at specific intervals such that the length of each optical fiber 30 can be limited to the aforementioned range. Further, as shown in FIG. 2, in a case where the band for curing the human body in accordance with the present invention is made in the form of a patch, the band is configured in such a manner that one or more silver wires 20′ and optical fibers 30 are repeatedly arranged onto a surface of the sheet member coated with an adhesive and the resultant band is cut several times in a direction perpendicular to a direction in which the silver wires and optical fibers are arranged such that it can be used as a band having a length of 0.1 cm to 2.0 cm. This is preferable in view of the manufacturing cost reduction.

Furthermore, as shown in FIG. 2, in a case where the silver is made in the form of the silver wire 20′, a band for curing the human body in accordance with the present invention is configured in such a manner that a protective film 50 is attached onto a surface of the sheet member 10 with an adhesive coated thereon and the silver wires 20′ and optical fibers 30 then coated thereon, such that the band is not entangled or distorted due to the adhesive component. The band can be attached to an affected part of the human body in the form of a patch such as a disposable patch or plaster by peeling off the protective film 50 before use. The protective film 50 is preferably made of a flexible plastic material, but the material is not limited thereto.

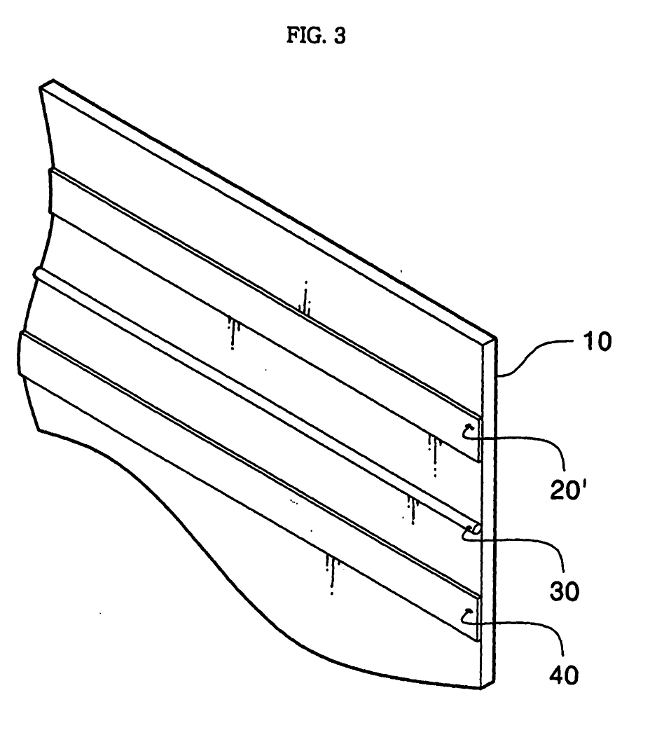

In addition, the band for curing the human body in accordance with the present invention can further comprise black lines 40 for absorbing light which are disposed on the surface of the sheet member 10 coated with the adhesive. As shown in FIGS. 2 and 3, the black lines 40 of the band in accordance with the present invention may be configured in such a manner that they can be arranged at regular intervals to be parallel with the silver wires 20′ and optical fibers 30. The white has the property of reflecting light, whereas the black has the property of absorbing light. The black lines 40 of the band in accordance with the present invention are used together with the white colors of other components, which can absorb or reflect light so as to keep the human body at its equilibrium state and then to keep the human body at its comfortable state or cure the human body. The black lines 40 may merely exhibit the black colors capable of absorbing light, and thus, the materials thereof need not be limited to a specific material. The black lines 40 are preferably made of graphite, iron oxide, activated carbon, or the mixture thereof, but the material is not limited thereto.

In use, the band for curing the human body in accordance with the present invention can be attached directly to the human body in the form of a patch such as a disposable patch or plaster and alternatively positioned adjacent to the affected part of the human body.

1. A band for curing a human body, comprising:
a flexible sheet member coated with an adhesive on a surface thereof;
silver attached to the surface of the sheet member coated with the adhesive; and
at least one optical fiber attached to the surface of the sheet member coated with the adhesive.

2. The band as claimed in claim 1, wherein the silver is silver foil, silver wire, silver fabric or the combination thereof.

3. The band as claimed in claim 2, wherein the optical fiber is positioned at regular intervals between the sheet member and the silver foil when the silver is made in the form of the silver foil.

4. The band as claimed in claim 2, wherein the optical fiber is alternately arranged on the sheet member at predetermined intervals with the silver wires when the silver is made in the form of the silver wires.

5. The band as claimed in claim 1, wherein a length of the optical fiber is within a range of 0.1 cm to 2.0 cm.

6. The band as claimed in claim 5, further comprising a black line disposed on the surface of the sheet member coated with the adhesive.

7. The band as claimed in claim 2, wherein a length of the optical fiber is within a range of 0.1 cm to 2.0 cm.

8. The band as claimed in claim 3, wherein a length of the optical fiber is within a range of 0.1 cm to 2.0 cm.

9. The band as claimed in claim 4, wherein a length of the optical fiber is within a range of 0.1 cm to 2.0 cm.

10. The band as claimed in claim 7, further comprising a black line disposed on the surface of the sheet member coated with the adhesive.

11. The band as claimed in claim 8, further comprising a black line disposed on the surface of the sheet member coated with the adhesive.

12. The band as claimed in claim 9, further comprising a black line disposed on the surface of the sheet member coated with the adhesive.
